(19) United States
(12) Patent Application Publication
(10) Pub. No.: US 2023/0054188 A1
(43) Pub. Date: Feb. 23, 2023

(54) PERITONEAL DIALYSIS SYSTEM AND CONTINUOUS GLUCOSE MONITORING

(71) Applicant: Bellco SRL, Mirandola (IT)

(72) Inventor: Roberto Carlos Chachero Gonzalez, Almazcara (ES)

(73) Assignee: Bellco SRL, Mirandola (IT)

(21) Appl. No.: 17/404,795

(22) Filed: Aug. 17, 2021

(51) Int. Cl. A61M 5/172; A61M 5/142; A61M 1/28

(52) U.S. Cl. CPC A61M 5/1723; A61M 5/14248; A61M 5/14276; A61M 1/282; A61M 2230/201; A61M 2205/3306

(57) ABSTRACT

Systems and method for monitoring the glucose concentration of a patient during a peritoneal dialysis session, and automated administration of a medication in response to the glucose concentration falling outside a specified range. The system includes a peritoneal dialysis system, a glucose sensor and at least one medication infusion pump. The peritoneal dialysis system includes a control system, and a peritoneal dialysate generation flow path fluidly connectable to a patient. The glucose sensor is in communication with the control system and positioned to continuously measure the glucose concentration of the patient during the peritoneal dialysis session. The control system can be programmed to provide automated administration of medication by the at least one medication infusion pump in response to changes in the glucose concentration of the patent during the hemodialysis session.

### FIELD

Systems, methods, and devices for continuous glucose monitoring and treatment of a patient during peritoneal dialysis treatment are provided.

### BACKGROUND

The mortality rate of end-stage renal disease (ESRD) patients who receive traditional peritoneal dialysis therapy is 6% per year, with an even higher mortality rate among diabetic patients. For example, a patient with diabetes mellitus and established renal involvement (i.e., with albuminuria and decreased glomerular filtering) has a mortality rate, at ten years, up to 10 times higher than a diabetic patient without kidney disease. Diabetic patients also have significant differences with other peritoneal dialysis patients in their demographic characteristics, complications, comorbidities, and treatment goals. A peritoneal dialysis patient having diabetes needs special management in most areas of peritoneal dialysis, such as peritoneal dialysis guidelines or diabetes control, etc. In many cases diabetic patients have significant variations in glucose figures during peritoneal dialysis, both hyperglycemia and symptomatic hypoglycemia, and other subsidiaries of immediate treatment.

However, the available systems and methods fail to monitor diabetic patients throughout a peritoneal dialysis session. The available systems and methods commonly fail to ensure patient stability and cannot automatically predict possible alterations and incidences of hypoglycemia and hyperglycemia of the patient's glucose during a peritoneal dialysis session. Although the available peritoneal dialysis systems and methods may attempt to monitor other patient or dialysate parameters, the systems and methods do not provide for the simultaneous and continuous monitoring of glucose levels during a peritoneal dialysis session. Furthermore, these known systems commonly fail to include a means for treating the instance of hypoglycemia or hyperglycemia in the first place. The available systems and methods do not use sensors that can monitor and/or control glucose levels. As a result, the patient, healthcare professionals and/or clinicians are required to perform manual controls during peritoneal dialysis to verify stability. The extra steps impose added economic cost, the use of consumables such as glucose strips, and require more time.

Hence, there is a need for peritoneal dialysis systems and methods that provide continuous monitoring of a glucose concentration of a patient. The need can include both diabetic and non-diabetic patients. There is a further need for automating the administration of medication by the peritoneal dialysis system in response to changing patient glucose levels during peritoneal dialysis. There is still a further need for automated and integrated glucose reading sensor and related alarms attendant to peritoneal dialysis. There is need for monitoring and controlling glucose for a patient during an entire course of peritoneal dialysis therapy. There is a need for automatically predicting possible alterations and incidences of hypoglycemia and hyperglycemia of the patient's glucose during peritoneal dialysis.

### SUMMARY

The problem to be solved is monitoring diabetic patients throughout a peritoneal dialysis session for possible alterations and incidences of hypoglycemia and hyperglycemia. The solution provides for a system that can include a system having a peritoneal dialysis system; the peritoneal dialysis system having a control system, and a peritoneal dialysate generation flow path fluidly connectable to a patient; a glucose sensor in communication with the control system; the glucose sensor positioned to continuously measure a glucose concentration of a patient during a peritoneal dialysis session; and at least one medication infusion pump in communication with the control system, the control system programmed to provide automated administration of a medication by the at least one medication infusion pump in response to a change in the glucose concentration being outside of a specified range during the peritoneal dialysis session.

In any embodiment, the glucose sensor can be a wearable glucose sensor.

In any embodiment, the glucose sensor can be positioned to measure a chemical composition of the skin of the patient to determine a glucose concentration.

In any embodiment, the glucose sensor can use Raman spectroscopy to measure the patient glucose level.

In any embodiment, the glucose sensor can be an implantable glucose sensor.

In any embodiment, the glucose sensor can include an electrode that is implanted under the skin of the patient to measure the glucose values.

In any embodiment, the at least one medication infusion pump can be a wearable medication infusion pump.

In any embodiment, the at least one medication infusion pump can be an implantable medication infusion pump.

In any embodiment, the at least one medication infusion pump can be integral the peritoneal dialysis system

In any embodiment, the control system can be programmed to continuously provide the glucose concentration to a user.

In any embodiment, the system can include a monitor, wherein the control system can be programmed to provide the glucose concentration on the monitor.

The features disclosed as being part of the first aspect of the disclosure can be in the first and third aspect of the disclosure, either alone or in combination.

The second aspect of the disclosure is drawn to a system. In any embodiment, the system can include a peritoneal dialysis system; the peritoneal dialysis system having a control system, and a peritoneal dialysate generation flow path fluidly connectable to the patient via a peritoneal dialysate fluid line; a glucose sensor in communication with the control system; wherein the glucose sensor is one of an implantable glucose sensor or a wearable glucose sensor, positioned to continuously measure a glucose concentration of the patient during a peritoneal dialysis session; and at least one medication infusion pump in communication with the control system, the control system programmed to communicate with the glucose sensor measuring the glucose level, to continuously provide a glucose concentration of the patient during the peritoneal dialysis session and to control automated administration of a medication by the at least one medication infusion pump in response to the glucose concentration being outside of a specified range.

In any embodiment, the system can include a monitor; the control system programmed to provide the glucose concentration of the patient to at least one of the patient and the monitor.

In any embodiment, the glucose sensor can be positioned to measure a chemical composition of the skin of the patient to determine a glucose concentration.

In any embodiment, the glucose sensor can use an electrode an electrode that is implanted under the skin of the patient to measure the glucose values.

In any embodiment, the at least one medication infusion pump can be one of a wearable medication infusion pump or an implantable medication infusion pump.

In any embodiment, the at least one medication infusion pump can be integral the peritoneal dialysis system.

In any embodiment, the control system can be programmed to provide automated administration of one of glucose or insulin by the at least one medication infusion pump in response to an incidence of hypoglycemia or an incidence of hyperglycemia.

In any embodiment, the control system can be programmed to provide an alert if the glucose concentration is outside of a specified range.

The features disclosed as being part of the second aspect of the disclosure can be in the first and third aspect of the disclosure, either alone or in combination.

The third aspect of the disclosure is drawn to a method of using the system of the first aspect. In any embodiment, the method can include the steps of continuously measuring a glucose concentration of a patient during a peritoneal dialysis session; receiving via the control system the glucose concentration of the patient during the peritoneal dialysis session; and providing automated administration of a medication by the at least one medication infusion pump in response to the glucose concentration being outside of a specified range.

In any embodiment, the method can include receiving the glucose concentration in the arterial patient line of the peritoneal dialysis system during the peritoneal dialysis session.

In any embodiment, the glucose sensor is one of a wearable glucose sensor or an implantable glucose sensor.

The features disclosed as being part of the third aspect of the disclosure can be in the first and second aspect of the disclosure, either alone or in combination.

### BRIEF DESCRIPTION OF DRAWINGS

FIG. 1 is a schematic of a system including a glucose sensor for sensing glucose concentration of a patient during a peritoneal dialysis session in accordance with any embodiment.

FIG. 2 is a block diagram of a system including a glucose sensor for sensing glucose concentration of a patient during a peritoneal dialysis session in accordance with any embodiment.

FIG. 3 illustrates a system including a glucose sensor for sensing glucose concentration of a patient during a peritoneal dialysis session in accordance with any embodiment.

FIG. 4 illustrates a block diagram of a system including a glucose sensor for sensing glucose concentration of a patient during a peritoneal dialysis session in accordance with any embodiment.

FIG. 5 illustrates a block diagram of a system including a glucose sensor for sensing glucose concentration of a patient during a peritoneal dialysis session in accordance with any embodiment.

FIG. 6 shows a peritoneal dialysate generation flow path including a glucose sensor for sensing for sensing glucose concentration of a patient during a peritoneal dialysis session in accordance with any embodiment.

FIG. 7 is a flow diagram illustrating a method in accordance with any embodiment described herein.

### DETAILED DESCRIPTION

Unless defined otherwise, all technical and scientific terms used generally have the same meaning as commonly understood by one of ordinary skill in the art.

The articles “a” and “an” are used to refer to one or to over one (i.e., to at least one) of the grammatical object of the article. For example, “an element” means one element or over one element.

The terms “administration,” administering,” “administer,” “delivering,” “deliver,” “introducing,” and “introduce” can be used, in context, interchangeably to indicate the introduction of a substance to a patient in need thereof, and can further mean the introduction of water, any agent or medication to a peritoneal dialysis circuit where such water, agent or medication will enter the blood of the patient by diffusion, transversal of a diffusion membrane or other means.

The term “automated administration” refers to delivering a substance to a patient without intervention by a person.

The term “chemical composition of the skin” refers to a concentration of one or more solutes in the skin of a patient.

The terms “communicate”, “communicating” and “communication” include but are not limited to, the connection of system electrical elements, either directly or wirelessly, using optical, electromagnetic, electrical, or mechanical connections, for data transmission among and between said elements.

The term “communication device” or “communication unit” refers to a device such as a telemetry system or any other alert system such as an audio feedback device, which can communicate monitoring results to a patient and/or a medical care personnel as needed. The term “communication device” in certain instances refers to a device which serves the purpose of sending information with transmission capabilities to another device which receives the information using receiving capabilities. The “communication device” can use electromagnetic, optical or acoustic means for signal transmission.

The term “comprising” includes, but is not limited to, whatever follows the word “comprising.” Thus, use of the term indicates that the listed elements are required or mandatory but that other elements are optional and may or may not be present.

The terms “concentration” and “solute concentration” refers to an amount of a solute dissolved in a given amount of a solvent.

The term “consisting of” includes and is limited to whatever follows the phrase the phrase “consisting of.” Thus, the phrase indicates that the limited elements are required or mandatory and that no other elements may be present.

The term “consisting essentially of” includes whatever follows the term “consisting essentially of” and additional elements, structures, acts or features that do not affect the basic operation of the apparatus, structure or method described.

The term “continuously” refers to an act that occurs repeatedly or constantly, or without interruption or interference, during a given period.

The terms “control”, “controlling”, or “controls” refers to the ability of one component to direct the actions of a second component.

A “control system” consists of combinations of components that act together to maintain a system to a desired set of performance specifications. The performance specifications can include sensors and monitoring components, processors, memory and computer components configured to interoperate.

The terms “determining” and “determine” refer to ascertaining a particular state of a system or variable(s).

The term “electrode” as used herein describes an electrical conductor used to make contact with a part of a fluid, a solid or solution. For example, electrical conductors can be used as electrodes to contact any fluid (e.g., dialysate) to measure the conductivity of the fluid or deliver or receive charge to the fluid. A “disc electrode” includes an electrode with an electrode head in the shape of a disc. A “rod electrode” refers to an electrode in the shape of a rod or cylinder, with one end functioning as an electrode head. A “sheet electrode” refers to an electrode with an electrode head in the shape of a sheet. The sheet can be square, rectangular, circular or other solid planar geometries. A “mesh electrode” refers to an electrode with a mesh electrode head, where a mesh is the same as that described for a mesh electrode. An “antenna electrode” refers to an electrode with an electrode head in the shape of an antenna, where antenna shape refers to a serpentine structure of conductive wires or strips. A “pin electrode” refers to a rod electrode with a small diameter. Other electrode and electrode head geometries can be considered.

The term “fluid” can be any substance without a fixed shape that yields easily to external pressure such as a gas or a liquid. Specifically, the fluid can be water containing any solutes at any concentration. The fluid can also be dialysate of any type including fresh, partially used, or spent.

The term “fluidly connectable”, “fluid connection,” “fluidly connectable,” “fluidically engage”, or “fluidically coupled” refers to the ability of providing for the passage of fluid, gas, or combination thereof, from one point to another point. The ability of providing such passage can be any connection, fastening, or forming between two points to permit the flow of fluid, gas, or combinations thereof. The two points can be within or between any one or more of compartments of any type, modules, systems, components, and rechargers.

The term “fluidly connected” refers to a particular state such that the passage of fluid, gas, or combination thereof, is provided from one point to another point. The connection state can also include an unconnected state, such that the two points are disconnected from each other to discontinue flow. It will be further understood that the two “fluidly connectable” points, as defined above, can form a “fluidly connected” state. The two points can be within or between any one or more of compartments, modules, systems, components, and rechargers, all of any type.

The term “glucose” refers to a sugar, also called dextrose. Glucose is one of a group of carbohydrates known as simple sugars (monosaccharides) having the molecular formula C₆H₁₂O₆.

The term “glucose concentration” refers to an amount of glucose dissolved in a given volume of solvent.

The term “glucose sensor” as used herein refers to a device which measures the glucose concentration in the blood or interstitial tissue of a patient. The glucose sensor may be used herein to measure a blood glucose level wherein four grams, or about a teaspoon, is monitored for normal function in a number of tissues. The glucose sensor may also be used herein to measure the glucose levels in the fluid that surrounds the cells of a patient's tissues, referred to as fluid interstitial. Typically, glucose passes first into the blood and then into the interstitial fluid.

The terms “implantable” or “implanted” describe a device, component or module intended to be totally or partially introduced, surgically or medically into a body, or by medical intervention that remains after the procedure.

An “incidence of hypoglycemia” is a condition of a patient having lower blood glucose levels than normal.

An “incidence of hyperglycemia” is a condition of a patient having higher blood glucose levels than normal.

The term “infusing” or to “infuse” a fluid refers to the movement of peritoneal dialysate into the peritoneal cavity of a patient.

The term “insulin” refers to a hormone produced in the pancreas, which regulates the amount of glucose in the blood.

The term “integral” refers to a component that is part of, or connected to, a larger system.

The terms “measuring,” to “measure,” or “measurement” refer to determining a state or parameter of a system or substance.

A “medication infusion pump” is a pump configured to deliver one or more medications into the body of a patient.

The term “memory” refers to any device for recording digital information that can be accessed by a microprocessor, such as RAM, Dynamic RAM, microprocessor cache, FLASH memory, or memory card.

A “monitor” is a user interface on which information can be digitally provided.

The term “outside of a specified range” refers to a value of a parameter that is not within a given range of values.

The term “patient” or “subject” refers to a member of any animal species, preferably a mammalian species, optionally a human. The subject can be an apparently healthy individual, an individual suffering from a disease, or an individual being treated for an acute condition or a chronic disease.

“Peritoneal dialysate” or “peritoneal dialysis fluid” is a dialysis solution to be used in peritoneal dialysis having specified parameters for purity and sterility. Peritoneal dialysate is not the same as dialysate used in peritoneal dialysis although peritoneal dialysate may be used in peritoneal dialysis.

A “peritoneal dialysate generation flow path” is a path used in generating dialysate suitable for peritoneal dialysis.

A “peritoneal dialysate generation system” refers to a collection of components used to generate peritoneal dialysate.

A “peritoneal dialysate fluid line” is a fluid line through which peritoneal dialysate can be infused into or drained from a patient.

The term “peritoneal dialysis” refers to a therapy wherein a dialysate is infused into the peritoneal cavity, which serves as a natural dialyzer. In general, waste components diffuse from a patient's bloodstream across a peritoneal membrane into the peritoneal dialysis solution via a concentration gradient. In general, excess fluid in the form of plasma water flows from a patient's bloodstream across a peritoneal membrane into the peritoneal dialysis solution via an osmotic gradient.

The term “peritoneal dialysis cycler” or “cycler” refers to components for movement of fluid into and out of the peritoneal cavity of a patient, with or without additional components for generating peritoneal dialysate or performing additional functions.

A “peritoneal dialysis session” is a set of peritoneal dialysis cycles performed over a time period as part of ongoing therapy. The peritoneal dialysis session can last a day or more, and can include any number of cycles,

The term “peritoneal dialysis system” refers to a set of components used to deliver peritoneal dialysis therapy.

The term “position” or “positioned” refers to a physical location of a component or system.

The term “programmable,” “programmed,” and the like as used herein refers to a device using computer hardware architecture and being capable of carrying out a set of commands, automatically.

The term “providing an alert” or to “provide an alert” refer to a system generating any type of audio, visual, or tactile cue of a particular state of a patient or system.

The term “pump” refers to any device that causes the movement of fluids or gases by the application of suction or pressure.

The terms “pumping,” “pumped,” or to “pump” refers to moving or flowing a fluid using a pump of any type known to those of ordinary skill in the art.

The term “Raman spectroscopy” refers to a molecular spectroscopic technique based on a light scatting process wherein light interacts with matter to provide a material's make up or characteristics.

The term “receiving” or to “receive” means to obtain information from any source.

The term “sensor,” which can also be referred to as a “detector” in certain instances, and as used herein can be any or combination of a mechanical transducer, converter, optical, electrical, or any other sensing modality that can detect, measure, or sense a physical or otherwise measurable quantity of a matter in a solution, liquid, gas, or combinations thereof. In any non-limiting embodiment, the sensor can measure chemicals, solute concentration, flow, density, content, or any other measurand and convert the obtained value into a signal of any type which can be read by an instrument, circuit, computer, processor, or any other suitable component or device.

The term “wearable” describes a device, component or module intended to be attached or in contact with a patient body but not implanted within the patient's body.

### Peritoneal Dialysis System Including Continuous Glucose Monitoring

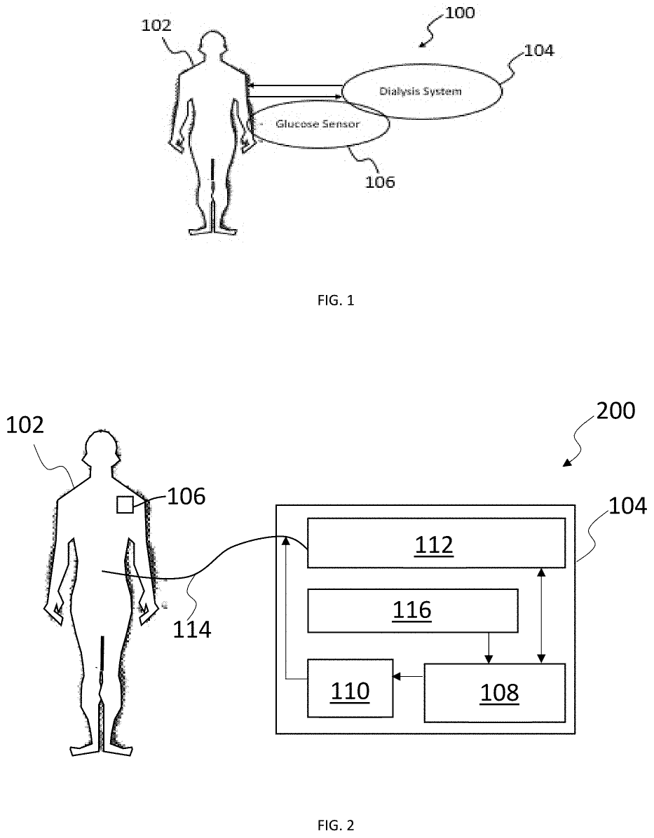

FIG. 1 shows a patient monitoring system operating in a continuous manner during a peritoneal dialysis session in which glucose levels, either blood or interstitial tissue glucose levels, are monitored and medications may be administered automatically in response to glucose levels outside a desired range. During a peritoneal dialysis session, the peritoneum, which is the lining of the abdomen, acts as a natural filter to remove waste products from the patient's blood. One goal during peritoneal dialysis, is to ensure that the patient's glucose concentration is within an acceptable range. In any embodiment, the peritoneal dialysis system and methods can identify out-of-range patient glucose concentrations. Typically, a patient's blood glucose level can range during or following a blood fluid removal session from about 100 mg/dL (5.55 mmol/L) to about less than 140 mg/dL (7.8 mmol/L). During a peritoneal dialysis session, glucose levels can sometimes dip further to about (<5.55 mmol/L). Such low glucose levels can be associated with increased mortality risk. Conditions that may affect a patient's glucose level during a peritoneal dialysis include nutritional deficiencies, reduced ability of the kidneys to form new glucose molecules, loss of glucose to the dialysate, decreased insulin clearance, and glucose diffusion into erythrocytes. These conditions may present during a peritoneal dialysis session as changes in glucose metabolism, insulin resistance/secretion/degradation and changes in drug metabolism to treat such.

The methods, systems and devices described herein may be used, in any embodiment, to determine an initial glucose level based on monitoring that occurs before a peritoneal dialysis session starts. Glucose levels, measured in either the blood or interstitial tissues, can change at any point during the peritoneal dialysis session. In any embodiment, the systems and methods can monitor the patient's glucose levels intermittently, periodically or continuously over the course of the peritoneal dialysis session. To minimize interference with the patient, the glucose sensing component(s) thereof, can be implanted or configured as a wearable device. Suitable glucose sensing components can be described to automatically detect and monitor glucose levels.

In any embodiment, the systems and methods can provide medication to the patient during peritoneal dialysis in response to sensed changes in glucose levels outside of a specified range. The delivery of medication can be automated to occur upon receiving certain specified glucose values or other monitored physiological measurements that recommend the delivery of a particular medicament.

In any embodiment, one or more sensors can be employed to detect glucose levels in the body of the patient. The glucose levels can be detected in a blood sample, on skin surface contact, via a measurement in interstitial tissue, or any other body compartment or combination of body compartments that can provide a measured or predicted value of glucose in the patient. For example, a blood glucose sensor that measures glucose concentration by direct contact with bodily fluids can be employed. Alternatively, a glucose sensor that measure glucose concentration by indirect contact with bodily fluids, can be employed. External or implantable glucose sensors can be used to measure the chemical composition of the skin to determine the amount of glucose and can optionally be used in conjunction with a sensor that takes measurements through direct contact with bodily fluids if necessitated. In any embodiment, the sensor can be replaced subsequent to its useful life. The output from any one or more combination of sensors of different types can be used together to develop a glucose level or glucose status of a patient. In any embodiment, more than one sensor can be employed for purposes of result confirmation and redundancy, which can improve reliability and accuracy. In any embodiment, the glucose sensor may be configured to accurately detect different ranges of glucose concentration.

FIG. 1 is a schematic showing an overview of a system 100 for sensing glucose and providing automated medication such as through the infusion of glucose and/or insulin, to a patient during a peritoneal dialysis session. The system 100 includes a peritoneal dialysis system 104, a glucose sensor 106 in communication with the peritoneal dialysis system 104 and a medication infusion pump 110 in communication with the peritoneal dialysis system 104. The glucose sensor 106 is positioned to continuously measure a glucose concentration of a patient 102 during a peritoneal dialysis treatment. The system 100, and more particularly, the medication infusion pump 110, is configured to provide automated administration of a medication in response to changing patient glucose levels outside of a desired range during the peritoneal dialysis session.

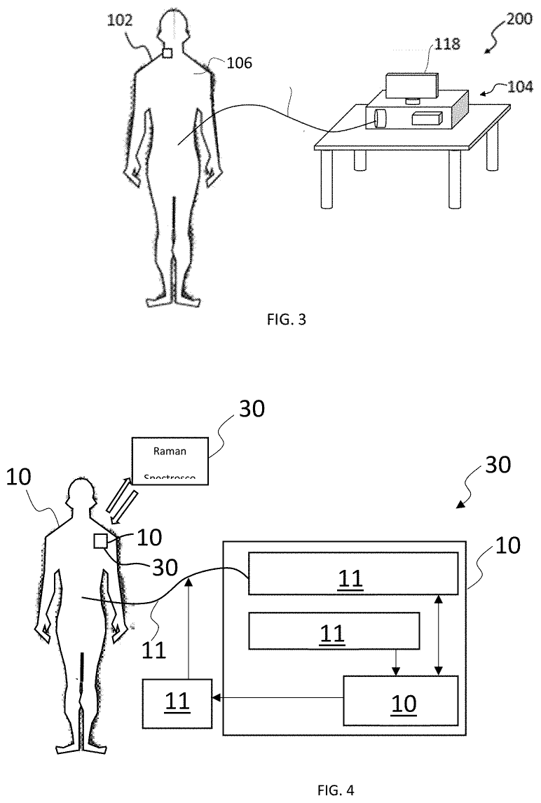

In FIG. 2 and FIG. 3, a system 200 includes the glucose sensor 106, the peritoneal dialysis system 104 including a control system 108, and a peritoneal dialysis cycler 112, including a portion of a peritoneal dialysate generation flow path (described presently), fluidly connectable to the patient 102 via a peritoneal dialysate fluid line 114, and the at least one medication infusion pump 110. In an alternate system, manual dialysis may be accomplished without the use of a cycler. The system 200 further includes a glucose sensor receiver 116 to provide continuous monitoring of the glucose level of a patient 102 during a peritoneal dialysis session. The glucose sensor receiver 116 is in communication with the glucose sensor 106 and the control system 108. The glucose sensor 106 is positioned to continuously measure a glucose concentration of the patient 102 during a peritoneal dialysis treatment. The control system 108 is programmed to provide automated administration of at least one medication by the at least one medication infusion pump 110 in response to changing patient glucose levels during the peritoneal dialysis session that fall outside of a desired range. One of ordinary skill will understand that features of the systems disclosed herein that have been previously described with regard to the schematic of FIG. 1 will be referenced throughout the figures as having the same reference number.

In FIG. 2 and FIG. 3, the at least one medication infusion pump 110 can be an integral part of the peritoneal dialysis system 104. The peritoneal dialysis system 104 can include additional sensors (described presently) for real-time monitoring of dialysis progress and patient status in biofeedback to help prevent any complications. In any embodiment, the peritoneal dialysis system 104 can provide real-time monitoring of peritoneal dialysis progress and patient status via multiple sensors, one of which can include the glucose sensor 106.

In the embodiment of FIGS. 2 and 3, the glucose sensor 106 can be a wearable glucose sensor, that is in contact with the patient's skin to obtain glucose data. More particularly, the glucose sensor 106 can be a non-invasive, wireless, continuous glucose monitoring system. The measurement would be performed on the patient's skin in a non-invasive manner. In the embodiment of FIGS. 2 and 3, the at least one medication infusion pump 110 is configured as an integral part of the peritoneal dialysis system 104. In this particular embodiment, the glucose sensor 106 is coupled to the control system 108 via a wireless coupling. In an alternate embodiment, the glucose sensor 106 is coupled to the control system 108 via a wired coupling. The glucose sensor 106 is configured to perform measurements of the glucose level of the patient 102 on a continual basis during the peritoneal dialysis session. During monitoring, the glucose sensor 106 obtains a glucose level measurement and a resulting signal is fed to the control system 108, which may or may not provide the obtained data on a monitor 118 or to a user. In response to obtained glucose data outside of a specified range, the peritoneal dialysis system 104, and more particularly, the at last one medication infusion pump 110 in response to a signal indicating glucose levels being outside a specified range, receives a signal to automatically infuse the medication to bring the glucose levels back into the specified range.

In any embodiment, the peritoneal dialysis system 104 can respond to patient diversity and clinical complexity by additionally monitoring biofeedback systems to recognize patient situations based on biochemical signals. The peritoneal dialysis system 104 can monitor and recognize clinically significant conditions monitored during a dialysis session. In any embodiment, the peritoneal dialysis system 104 can check for intra-dialysis complications. In any embodiment, the system 200 can include a means to check a patient's body temperature at a preset value according to medical prescription (isothermal or hypothermal method). In any embodiment, the peritoneal dialysis system 104 can control dialysis treatment, reducing the risk of possible hypotensive events for the patient. In any embodiment, the peritoneal dialysis system 104 can modulate dialysate conductivity according to personalized profiles. By simulating the intradialytic kinetics of the solute and fluid exchanges, the peritoneal dialysis system 104 can control the plasma sodium concentration and maintains the osmolar equilibrium, to prevent primary and post dialysis clinical complications, such as hypotension and disequilibrium syndrome.

The glucose sensor 106 can be positioned to continuously measure a glucose concentration of the patient 102 during a peritoneal dialysis treatment. The control system 108 is programmed to provide automated administration of at least one medication by the at least one medication infusion pump 110 in response to changing patient glucose levels during the peritoneal dialysis session. Notably, the glucose sensor 106 can be disposed separate and apart from the peritoneal dialysis system 104. In any embodiment, the glucose sensor 106 is in communication with the control system 108 via a wired or wireless coupling to provide continuous monitoring of the glucose level of a patient 102 during a peritoneal dialysis session.

The glucose sensor 106 can be configured to perform continuous measurements of the glucose level of the patient on a continual basis during the peritoneal dialysis session. During monitoring, the glucose sensor 106 obtains a glucose level measurement and a resulting signal can be fed to the control system 108, which may or may not provide the obtained data on a monitor. In response to obtained glucose data outside of a specified range, the peritoneal dialysis system 104, and more particularly, the at last one medication infusion pump 110 in response to a signal indicating glucose levels being outside a specified range, receives a signal to automatically infuse a medication to bring the glucose levels back into the specified range.

As illustrated, the glucose sensor 106 is disposed separate and apart from the peritoneal dialysis system 104. The glucose sensor 106 is in communication with the control system 108 via the glucose sensor receiver 116 to provide continuous monitoring of the glucose level of a patient 102 during a peritoneal dialysis session.

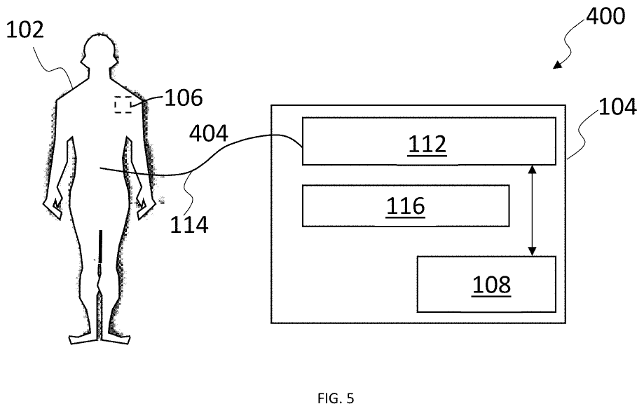

In FIG. 4, a system 300 includes the glucose sensor 106, the peritoneal dialysis system 104 having a control system 108, and a peritoneal dialysis cycler 112, including a portion of a peritoneal dialysate generation flow path (described presently), fluidly connectable to the patient 102 via a peritoneal dialysate fluid line 114, and at least one medication infusion pump 110. In an alternate system, manual dialysis may be accomplished without the use of a cycler. The system 300 further includes the glucose sensor 106 in communication with the control system 108. The glucose sensor 106 can be positioned to continuously measure a glucose concentration of the patient 102 during a peritoneal dialysis treatment. The control system 108 is programmed to provide automated administration of at least one medication by the at least one medication infusion pump 110 in response to changing patient glucose levels during the peritoneal dialysis session. Similar to the embodiments of FIG. 2-4, the glucose sensor 106 can be disposed separate and apart from the peritoneal dialysis system 104. As illustrated, the glucose sensor 106 can be in communication with the control system 108 via a glucose sensor receiver 116 to provide continuous monitoring of the glucose level of a patient 102 during a peritoneal dialysis session.

In the embodiment of FIG. 4, the glucose sensor 106 can be a wearable glucose sensor 304, that is in contact with the patient's skin to obtain blood glucose data. More particularly, the blood glucose sensor 106 can be a non-invasive, wireless, continuous glucose monitoring system. In any embodiment, the glucose sensor 304 can utilize Raman spectroscopy 306 to measure the chemical composition of the patient's skin to determine the level of glucose. This type of sensor measures fat tissue, protein, collagen, and glucose molecules. The measurement would be performed on the patient's skin in a non-invasive manner and the data transmitted to a display of the peritoneal dialysis monitor. In the embodiment of FIG. 4, the at least one medication infusion pump 110 is configured separate from the peritoneal dialysis system 104, and in communication with the control system 108. In this particular embodiment, the glucose sensor 106 is coupled to the control system 108 via a wireless coupling. In an alternate embodiment, the glucose sensor 106 is coupled to the control system 108 via a wired coupling. The glucose sensor 106 is configured to perform measurements of the glucose level of the patient on a continual basis during the peritoneal dialysis session. During monitoring, the glucose sensor 106 obtains a glucose level measurement and a resulting signal is fed to the control system 108, which may or may not provide the obtained data on a monitor or to a user. In response to obtained glucose data outside of a specified range, the peritoneal dialysis system 104, and more particularly, the at last one medication infusion pump 110 in response to a signal indicating glucose levels being outside a specified range, receives a signal to automatically infuse the medication to bring the patient's glucose levels back into the specified range.

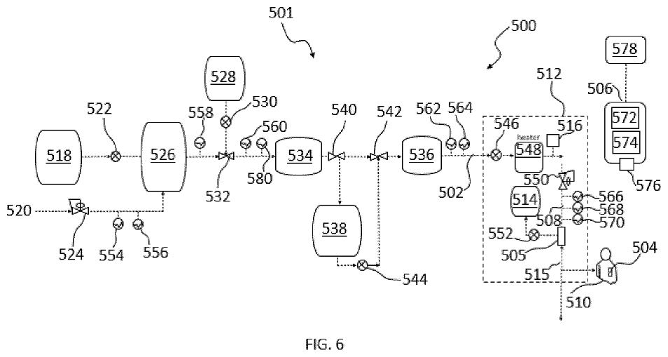

Referring now to FIG. 5, a system 400 includes the glucose sensor 106, and the peritoneal dialysis system 104 having a control system 108, and a peritoneal dialysis cycler 112, including a portion of a peritoneal dialysate generation flow path (described presently), fluidly connectable to the patient 102 via a peritoneal dialysate fluid line 114, and at least one medication infusion pump 110. In an alternate system, manual dialysis may be accomplished without the use of a cycler. The system 400 further includes a glucose sensor 106 in communication with the control system 108. The glucose sensor 106 can be positioned to continuously measure a glucose concentration of the patient 102 during a peritoneal dialysis treatment. The control system 108 can be programmed to provide automated administration of at least one medication by the at least one medication infusion pump 110 in response to changing patient glucose levels during the peritoneal dialysis session. Similar to the embodiment of FIGS. 2-4, the glucose sensor 106 is disposed separate and apart from the peritoneal dialysis system 104. As illustrated, the glucose sensor 106 can be in communication with the control system 108 via a glucose sensor receiver 116 to provide continuous monitoring of the glucose level of a patient 102 during a peritoneal dialysis session.

In the embodiment of FIG. 5, the glucose sensor 106 can be an implantable blood glucose sensor 404, that can be implanted subcutaneously under the patient's skin to obtain blood glucose data. Alternatively, the glucose sensor 106 can be a partially implantable blood glucose sensor 404 wherein the glucose sensor 106 is configured to include an electrode that is inserted just under the skin of the patient 102 to measure the glucose values. In any embodiment, the glucose sensor 404 can be the Medtronic ENLITE device. Such electrodes, and components of sensors employing such electrodes, are known in the art and may be employed, or modified to be employed, for use in the monitoring described herein. Generally similar to the previous embodiment, in the embodiment of FIG. 5, the at least one medication infusion pump 110 can be configured separate from the peritoneal dialysis system 104. In any embodiment, the at least one medication infusion pump 110 can be integrally formed with the glucose sensor 404 or work in conjunction with a link transmitter, e.g., Medtronic Guardian™ 2 or Guardian™ REAL-Time System, wherein glucose information is sent directly to an insulin pump, e.g., MiniMed™ insulin pump adapted to the peritoneal dialysis monitor. For example, in an embodiment, the glucose sensor 106 would be placed on the patient and connected wirelessly to a MiniMed™ pump during each dialysis session. A typical sensor has a useful life of six (6) days and would be replaced at the end of this time with a new sensor. In another embodiment, a luer-lock style connector could be used to fit the peritoneal dialysate fluid line 114 and infuse the medication through the fluid line. In any embodiment, the infusion pump 110 and the glucose sensor 404 can be compatible or paired to otherwise work in concert with any suitable smart pump technology systems during dialysis therapy. In an alternate embodiment, the peritoneal dialysis monitor's software may be adapted to provide reading data from a subcutaneous sensor and transmit the data via wireless connection.

In any embodiment, the glucose sensor 106 can be coupled to the control system 108 via a wireless coupling. In an alternate embodiment, the glucose sensor 106 can be coupled to the control system 108 via a wired coupling. The glucose sensor 106 can be configured to perform measurements of the glucose level of the patient on a continual basis during the peritoneal dialysis session. During monitoring, the glucose sensor 106 obtains a glucose level measurement and a resulting signal is fed to the control system 108, which may or may not provide the obtained data on a monitor or to a user. In response to obtained glucose data outside of a specified range, the peritoneal dialysis system 104, and more particularly, the at last one medication infusion pump 110 in response to a signal indicating glucose levels being outside the specified range, receives a signal to automatically infuse the medication to bring the glucose levels back into the specified range.

In any of the systems previously described, the user can continuously monitor a patient's glucose values on the peritoneal dialysis monitor screen, schedule alarms with desired maximum and minimum values, and activate acoustic warnings when the patient is undergoing treatment. The system disclosed herein continuously monitors certain parameters (in this case the glucose of a diabetic patient on peritoneal dialysis). The system performs a continuous measurement of glucose throughout the peritoneal dialysis treatment, these measurements may be displayed in the peritoneal dialysis monitor interface, indicating the initial glucose values of the patient and the constant values during treatment, can also offer an evolution graph throughout the peritoneal dialysis session. The display has user configurable parameters with maximum and minimum alarm limits with visual and acoustic alarms. In this way the user can anticipate an incident of hyperglycemia or hypoglycemia during peritoneal dialysis treatment, thus achieving more hemodynamic stability and treatment.

In any of the systems previously described, the system provides the automatic administration of medication if the patient's glucose values exceed configured safety parameters ranges. To provide for the automatic administration of medication, the system can be preprogrammed with desired parameters, such as g/dl of glucose to be administered, an alarm parameter to start glucose administration, program the insulin units to be administered when the patient's values exceed the programmed maximum glycemic range parameter, etc.

Any of the glucose sensors 106 of the systems 100, 200, 300, 400 disclosed herein may be calibrated prior to sensing (in any condition mimicking the final sensor environment) with a known glucose concentration. The sensors can be recalibrated subsequent to placement relative to the patient. For example, blood glucose level can be measured external to the patient, e.g., via blood draws, and results of the external monitoring can be communicated to the sensor by receiving input, e.g., from the user or healthcare providers. Thus, the sensor can recalibrate based on the input regarding the external measurements if necessitated. Alternatively, or in addition, the sensor may have an internal reference built in. In cases where the sensor outputs raw data to an external device, the external device may be calibrated to interpret the raw data from the sensor with regard to input regarding the external measurements.

FIG. 6 illustrates a system 500 for sensing glucose and providing automated medication such as through the infusion of glucose and/or insulin, to a patient during a peritoneal dialysis session. The system 500 defines a peritoneal dialysate system 501 including a peritoneal dialysate generation flow path 502 and a control system 506, in communication with a glucose sensor 504, and at least one medication infusion pump 505, as previously described. The glucose sensor 504 is positioned to continuously measure a glucose concentration of a patient during a peritoneal dialysis treatment. The control system 506 is programmed to receive a signal from glucose sensor 504 and determine a glucose level of the patient undergoing peritoneal dialysis. The system 500, as illustrated, includes a combined peritoneal dialysate effluent line and infusion line, referred to herein as a peritoneal dialysate fluid line 508. The peritoneal dialysate fluid line 508 is in fluid communication with a catheter (not shown) having a single channel used for both filling and removal of the peritoneal dialysate fluid from a peritoneal cavity of a patient 510. One of skill in the art will understand that separate effluent and infusion lines can be used. The peritoneal dialysis system 500 is configured to direct the peritoneal dialysate fluid to the peritoneal dialysate fluid line 508 and patient 510.

The peritoneal dialysis system 501 can be embodied as a manual peritoneal dialysis system or an automated system including an integrated peritoneal dialysis cycler 512 wherein the peritoneal dialysis cycler 512 includes the peritoneal dialysate fluid line 508 and a portion of the peritoneal dialysate generation flow path 502. Alternatively, the peritoneal dialysis cycler 512 can be nonintegrated with the peritoneal dialysate generation flow path 502, whereby the peritoneal dialysate can be prepared off-line and provided to the peritoneal dialysis cycler 512. In an alternate embodiment, the system may operate manually and not include a cycler. The control system 506 can be a separate device or can be a part of the peritoneal dialysis cycler 512, whether integrated or nonintegrated.

The peritoneal dialysate fluid line 508 can be fluidly connected to a waste reservoir 514 to collect effluent peritoneal dialysate fluid. Optionally, a diverted flow path, such as the drain line 515, can be in fluid communication with the peritoneal dialysate fluid line 508 for analysis of the peritoneal dialysate fluid outside of the peritoneal dialysis cycler 512 and/or directing of the peritoneal dialysate fluid into a drain line 515 if the fluid characteristics in the fluid manifold are outside of a predetermined range. A valve (not shown) in the peritoneal dialysis cycler 512 can divert fluid from the peritoneal dialysate fluid line 508 to the drain line 515 to provide determination of fluid characteristics of the peritoneal dialysate fluid outside of the peritoneal dialysis cycler 512 continuously or at specific intervals and in predetermined amounts.

A pump (not shown) can provide an additional driving force for moving peritoneal dialysate through the diverted flow path. A similar analysis can be conducted on the generated peritoneal dialysate by diverting a volume of generated peritoneal dialysate into the diverted path (not shown). Analysis of the generated peritoneal dialysate can serve as a quality check on the newly generated peritoneal dialysate by comparing sensed values to known values of the dialysate. Analysis of the newly generated dialysate can also be used by the system for self-learning or machine learning to adjust the dialysate composition to a precision beyond the capabilities of known systems.

Alternatively, or additionally, the system 500 can include a sampling port 516. The sampling port 516 can be fluidly connected to the peritoneal dialysate fluid line 508. The sampling port 516 can alternatively be fluidly connected to the diverted flow path. The sampling port 516 can be covered by a pierceable septum. A user can insert a needle or syringe through the pierceable septum to draw out a portion of the peritoneal dialysate in the peritoneal dialysate fluid line 508 or diverted flow path. The pierceable septum can re-seal after removal of the needle or syringe to avoid contamination of the peritoneal dialysate.

When used with the integrated peritoneal dialysis cycler 512, the peritoneal dialysate generation flow path 502 can further include a water source, such as a water source 518, one or more water purification modules 526, a concentrate source 528, a sterilization module, such as one or more sterilization module filter 534 and/or UV light source 536, and the peritoneal dialysis cycler 512. The water source 518, the water purification module 526, the concentrate source 528, the sterilization module filter 534 and/or UV light source 536, and peritoneal dialysis cycler 512 can be fluidly connectable to the peritoneal dialysate generation flow path 502. The peritoneal dialysate generation flow path 502 can be fluidly connected to the peritoneal dialysate fluid line 508 to infuse peritoneal dialysate into the peritoneal cavity of the patient 510.

In an alternate embodiment, either additionally, or as an alternative to a water source 518, the system 500 can use a direct connection to a water source 520. A system pump 522 can control the movement of fluid through the peritoneal dialysate generation flow path 502. If a direct connection to a water source 520 is used, a pressure regulator 524 can ensure that an incoming water pressure is within a predetermined range. The water source 520 can be a non-purified water source, such as tap water, wherein the water from the water source 520 can be purified by the system. A non-purified water source can provide water without additional purification, such as tap water from a municipal water source, water that has undergone some level of purification, but does not meet the definition of “purified water” provided, such as bottled water or filtered water. The water source can contain water meeting the WHO drinkable water standards provided in Guidelines for Drinking Water Quality, World Health Organization, Geneva, Switzerland, 4th edition, 2011. Alternatively, the water source 520 can be a source of purified water, meaning water that meets the applicable standards for use in peritoneal dialysis without additional purification.

The system pumps the fluid from water source 518 or 520 through a water purification module 526 to remove chemical contaminants in the fluid in preparation for creating the peritoneal dialysate. The system pumps water from the water source to a water purification module to remove chemical contaminants in the fluid in preparation of the dialysate. The water purification module 526 can be a sorbent cartridge containing anion and cation exchange resins and/or activated carbon.

After the fluid passes through the water purification module 526, the fluid is pumped to a concentrate source 528, where necessary components for carrying out peritoneal dialysis can be added from the concentrate source 528. The concentrate source 528 can contain one or more solutes for generating the peritoneal dialysate from purified water. In an embodiment, the concentrate source 528 is a dextrose concentrate source containing solid dextrose, wherein adding water to the dextrose solute generates the dextrose concentrate.

The one or more solutes in the concentrate source 528 are utilized to create a peritoneal dialysis fluid that matches a dialysis prescription. A concentrate pump 530 and concentrate valve 532 in communication with the processor or computing unit control the movement of concentrates from the concentrate source 528 to the peritoneal dialysate generation flow path 502 in a controlled addition. Alternatively, the concentrate valve 532 can be a hose T or backflow restricting hose T. The concentrates added from the concentrate source 528 to the peritoneal dialysate generation flow path 502 can include components required for use in peritoneal dialysate. One of skill in the art will understand that any number of concentrate sources can be used, each containing concentrates of one or more substances. For example, the concentrate source 528 can include any number of concentrates combined or in separate concentrate sources. One or more osmotic agent sources can be included in addition to a single ion concentrate source. Alternatively, multiple ion concentrate sources can be used with each ion concentrate in a separate concentrate source. Any combination of concentrates in any number of concentrate sources can be used. The concentrate sources can infuse each particular concentrate to provide an infused ion concentration that is lower than a prescribed amount for a particular patient. One desired outcome can be to provide a concentration for a particular ion that is lower than a patient's pre-dialysis ion concentration. Additionally, if multiple ion sources are to be delivered by a concentrate source, the present system can selectively dilute a desired ion while maintaining concentration levels for other ions. Hence, the present disclosure can avoid adjusting down every ion insofar as an added diluent may adversely affect concentrations of ions already in a normal range.

Upon addition of solutes from the concentrate source 528, the fluid in the peritoneal dialysate generation flow path 502 can contain all the necessary solutes for peritoneal dialysis. The peritoneal dialysate should reach a level of sterility for peritoneal dialysis such that patients will not contract an infection due to bacteria or other pathogens in fluid used for peritoneal dialysate. The system 500 can pump the fluid to a sterilization module for sterilizing the peritoneal dialysate prior to infusion into the patient 510. As shown in FIG. 6, the sterilization module can include one or more of a sterilization module filter 534 and a UV light source 536, or any combination thereof. In any embodiment, the sterilization module filter 534 can include a microbial filter, nanofilter, or any filter device inhibiting passage of microfibers or fragments of microbes such as endotoxins in the peritoneal dialysate while allowing the passage of the peritoneal dialysate. In any embodiment, the UV light source 536 can be positioned at any location in the peritoneal dialysate generation flow path 502, including upstream of the sterilization module filter 534, downstream of the sterilization module filter 534, or disposed between multiple filters in a multi-filter system. The sterilization module can be any component or set of components capable of sterilizing the peritoneal dialysate.

The generated peritoneal dialysate can be pumped directly to the integrated peritoneal dialysis cycler 512 for immediate infusion into a patient 510. Alternatively, the dialysate can be pumped to an optional dialysate container 538 as a pre-prepared bolus of solution for storage until ready for use by a patient 510. A plurality of valves 540, 542 can control the movement of fluid to either the dialysate container 538 or the integrated peritoneal dialysis cycler 512. Stored dialysate in dialysate container 538 can be pumped as needed to back into the peritoneal dialysate generation flow path 502 by a pump 544 through valve 542. The dialysate container 538 can store enough peritoneal dialysate for a single infusion of peritoneal dialysate into the patient 510, or enough peritoneal dialysate for multiple or continuous infusions into one or multiple patients.

In an embodiment, the generated peritoneal dialysate can be pumped to a valve (not shown) that can control movement of the peritoneal dialysate to multiple flow options. The peritoneal dialysate can be pumped to the integrated peritoneal dialysis cycler 512 or diverted for use with a non-integrated external cycler (not shown) or to a dialysate container (not shown). All various pumping/diversion options can be performed contemporaneously or selectively. Alternative valve and pump configurations for performing the same functions are contemplated by the present invention. For example, a direct connection to an external cycler can use any type of connector known in the art. The connectors can be single-use or reusable connectors and should provide for sterile transfer of fluids. The connectors should preferably be closed connectors, to avoid contact between the fluids and the external environment.

The integrated peritoneal dialysis cycler 512 can include a metering pump 546 for metering peritoneal dialysate into the peritoneal cavity of the patient 510. A heater 548 heats the peritoneal dialysate to a desired temperature prior to infusion into the patient 510. A pressure regulator 550 ensures the peritoneal dialysate pressure is within a predetermined range safe for infusion into the patient 510. The metering pump 546 can use any safe pressure for infusing fluid into the patient 510. Generally, the pump pressures are on average set at ±10.3 kPa or 77.6 mmHg. If there is no fluid flow, the maximum pressure can increase to ±15.2 kPa or 113.8 mmHg for a short period, such as less than 10 seconds. The peritoneal dialysate is infused into the peritoneal cavity of the patient 510 through the peritoneal dialysate fluid line 508. After a dwell period, the peritoneal dialysate is drained from the patient 510 through the peritoneal dialysate fluid line 508, or a separate effluent line (previously described). A pump 552 provides a driving force for removing the peritoneal dialysate from the patient 510. The optional waste reservoir 514 can be included to store the used peritoneal dialysate for disposal. Alternatively, the peritoneal dialysate fluid line 508 can be directly connected to the drain line 515 for direct disposal. The waste reservoir 514 can be any size, including between 12 and 20 L. For patients requiring a higher drainage, a drain manifold can be included for connecting multiple waste reservoirs.

The glucose sensor 504 may be configured according to any of the embodiments previously described. The glucose sensor 504 performs measurements of the glucose level of the patient 510 on a continual basis during the peritoneal dialysis session. During monitoring, the glucose sensor 504 obtains a glucose level measurement and a resulting signal is fed to the control system 506, which may or may not provide the obtained data on a monitor or to a user. In response to obtained glucose data outside of a specified range, the peritoneal dialysis system 500, and more particularly, the at last one medication infusion pump 505 in response to a signal indicating glucose levels being outside the specified range, receives a signal to automatically infuse the medication to bring the glucose levels back into the specified range. As previously described in the embodiments of FIGS. 2-5, the at last one medication infusion pump 505 can be a separate device or can be a part of the peritoneal dialysis cycler 512, whether integrated or nonintegrated.

Various additional sensors may be utilized by the peritoneal dialysis system 500 to ensure that one or more fluid characteristics of the generated peritoneal dialysate fluid is within predetermined parameters. The additional sensors can be fluidly connected to one or more of the peritoneal dialysate generation flow path 502 and the peritoneal dialysate fluid line 508. The one or more additional sensors can be separate sensors or one or more combined sensors. In an embodiment, one or more additional sensors can be external to the peritoneal dialysis cycler 512. The system an also include duplication of analysis with duplicated sensors in multiple locations. Duplication of the analysis allows calibration of the sensors and acts as a safety check to ensure the sensors are properly functioning. The duplicated sensors can be attached to the peritoneal dialysis cycler 512 or in a standalone system.

The one or more additional sensors may include a flow meter 554 to ensure the incoming water is at a correct flow rate, while a pressure sensor 556 can ensure the incoming water is at an appropriate pressure. A conductivity sensor 558 can be used to ensure that the water exiting the water purification module 526 has been purified to a level safe for use in peritoneal dialysis. A conductivity sensor 560 can ensure the conductivity of the dialysate after the addition of concentrates from concentrate source 528 is within a predetermined range. A pH sensor 580 can ensure the pH of the peritoneal dialysate is within a predetermined range. After passing through the sterilization module including UV light source 536, a pH sensor 562 and a conductivity sensor 564 can be used to ensure that no changes in the pH or conductivity have occurred during purification or storage of the dialysate in the dialysate container 538. The integrated peritoneal dialysis cycler 512 further may include a flow meter 566, a pressure sensor 568 and a temperature sensor 570 to ensure that the dialysate being infused into the patient 510 is within a proper flow rate, pressure, and temperature range. One of skill in the art will understand that alternative or additional sensing methods can be used, and any sensor or method known in the art can be incorporated.

The control system 506 can include one or more processors 572, memory 574, and one or more input/output interfaces 576. One of ordinary skill in the art will recognize that the memory 574 can include long-term memory and operating memory, and/or memory serving as both long-term memory and operating memory. The memory 574 can be a machine-readable storage medium. The memory 574 can be in communication with the processor 572 and store instructions that when executed perform any of the methods of the present disclosure. The input/output interface(s) 576 can include an input port to receive information from the glucose sensor 504 and any of one or more additional sensors, and an output interface to output data to a user, such as an alert notification 578 regarding the sensed data. The processor 572 can be in communication with the glucose sensor 504 and any of one or more additional sensors and store data received from the glucose sensor 504 and any of one or more additional sensors in the memory 574. As with all features of the present application, intervening components, such as the input/output interface 576, can be present between the processor 572 and the glucose sensor 504 and any of one or more additional sensors. The control system 506 can be a stand-alone device independent of the peritoneal dialysis cycler 512 or can be a part of the peritoneal dialysis cycler 512. The control system 506 can be a remote device in network communication with the glucose sensor 504 and any of one or more additional sensors, such as via the internet. In an embodiment, the dialysis system 500 can include a user interface (not shown) in communication with the control system 506, allowing the patient 510 to direct one or more functions of the system.

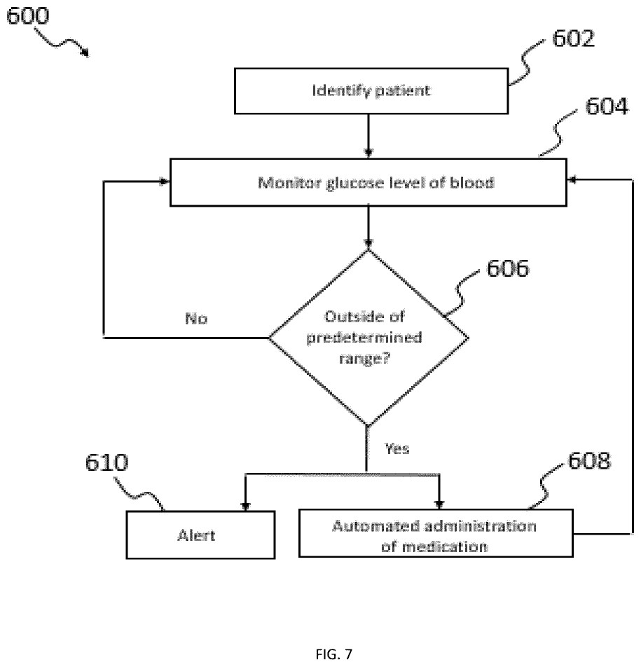

Referring now to FIG. 7, a depicted method 600 includes steps for identifying, selecting, or diagnosing a patient for which a peritoneal dialysis session is indicated in a step 602 and monitoring glucose levels of the patient in a step 604. The monitoring step 604 may be continuous and may employ one or more glucose sensors as previously described. The method 600 in FIG. 7 can include determining whether the glucose level is out of range in step 606 based on data acquired during the monitoring step 604. For example, a determination step 606 can be made as to whether the glucose level crossed a threshold (e.g., a ceiling or floor). Suitable thresholds or ranges may be stored in, for example, a look-up table in memory of a sensor device, the control system, or other suitable device for purposes of determining whether the glucose level is out of range in a step 606 based on data acquired during the monitoring. If the glucose level is determined to be within range, monitoring step 604 may continue. If the glucose level is determined to be out of range (e.g., cross a threshold), automated administration of medication to bring the glucose levels back into the specified range is performed in step 608. Subsequent to the administration of the medication, monitoring step 604 can include determining whether glucose levels are in a specified range.

In addition, as a result of the monitoring step 604, when the glucose level is determined to be out of range, any suitable alert may be issued in step 610. The alert may be a tactile cue, such as vibration or audible alarm, generated by a sensor or a device in communication with the sensor, such as the peritoneal dialysis system. The alert may provide the patient and/or healthcare provider with notice that medical attention should be sought. The alert may also provide information to the user or healthcare provider regarding the nature of the health issue (e.g., glucose level out of range) for which the alert in step 610 was issued. The sensor or the device in communication with the sensor may alert the patient and/or healthcare provider by transmitting the alert or related information over the internet, a telephone network, via a monitor, or other suitable network or component to a device in communication with the patient and/or healthcare provider.

As indicated above, sensors for monitoring patient physiological parameters may be, or may have components that are, contained within the peritoneal dialysis system, implantable or wearable. In embodiments, multiple sensors may be connected via telemetry, body bus, or the like. The connected sensors may be of the same or different type. Such connected sensors may be placed (e.g., internal or external) for purposes of monitoring at various locations of the patient's body.

Monitoring may alternatively or additionally include receiving patient or physician feedback regarding the patient's state. For example, the patient may indicate a point in time when nausea begins, which often happens when glucose levels are too high. The system may include an input, such as a keyboard or touch screen display for entering such data. Alternatively, a separate device such as a patient programmer, laptop computer, tablet computer, personal data assistance, smart phone or the like may be used to input the data; or the like.

The disclosed system for monitoring glucose levels of a patient during a peritoneal dialysis system and the automatic administration of medication during the session to address glucose levels outside of a specified range allows the diabetic patient to be monitored throughout the peritoneal dialysis session, ensuring patient stability and allows for predicting possible alterations and incidences (hypoglycemia, hyperglycemia) of the patient during the peritoneal dialysis session.

The particular embodiments disclosed above are illustrative only, and may be modified and practiced in different but equivalent manners apparent to those skilled in the art having the benefit of the teaching provided herein. Furthermore, no limitations are intended with respect to the details of construction, or the design shown herein, other than as described in the claims below. One skilled in the art will understand that various combinations and/or modifications and variations can be made in the systems and methods depending upon the specific needs for operation. Features illustrated or described as being part of an aspect as described herein can be used in any other aspect as described herein, either alone or in combination.

1. A system, comprising:
a peritoneal dialysis system; the peritoneal dialysis system comprising a control system, and a peritoneal dialysate generation flow path fluidly connectable to a patient;
a glucose sensor in communication with the control system; the glucose sensor positioned to continuously measure a glucose concentration of a patient during a peritoneal dialysis session; and
at least one medication infusion pump in communication with the control system,
the control system programmed to provide automated administration of a medication by the at least one medication infusion pump in response to a change in the glucose concentration being outside of a specified range during the peritoneal dialysis session.

2. The system of claim 1, wherein the glucose sensor is a wearable glucose sensor.

3. The system of claim 2, wherein the glucose sensor is positioned to measure a chemical composition of the skin of the patient to determine a glucose concentration.

4. The system of claim 3, wherein the glucose sensor uses Raman spectroscopy to measure the patient glucose level.

5. The system of claim 1, wherein the glucose sensor is an implantable glucose sensor.

6. The system of claim 5, wherein the glucose sensor comprises an electrode that is implanted under the skin of the patient to measure the glucose values.

7. The system of claim 1, wherein the at least one medication infusion pump is a wearable medication infusion pump.

8. The system of claim 1, wherein the at least one medication infusion pump is an implantable medication infusion pump.

9. The system of claim 1, wherein the at least one medication infusion pump is integral the peritoneal dialysis system.

10. The system of claim 1, the control system programmed to continuously provide the glucose concentration to a user.

11. The system of claim 1, further comprising a monitor; the control system programmed to provide the glucose concentration on the monitor.

12. The system of claim 1, the control system programmed to provide an alert if the glucose concentration is outside of a specified range.

13. A system, comprising:
a peritoneal dialysis system; the peritoneal dialysis system comprising a control system, and a peritoneal dialysate generation flow path fluidly connectable to the patient via a peritoneal dialysate fluid line;
a glucose sensor in communication with the control system; wherein the glucose sensor is one of an implantable glucose sensor or a wearable glucose sensor, positioned to continuously measure a glucose concentration of the patient during a peritoneal dialysis session; and
at least one medication infusion pump in communication with the control system,
the control system programmed to communicate with the glucose sensor measuring the glucose level, to continuously provide a glucose concentration of the patient during the peritoneal dialysis session and to control automated administration of a medication by the at least one medication infusion pump in response to the glucose concentration being outside of a specified range.

14. The system of claim 13, the control system further comprising a monitor, the control system further programmed to provide the glucose concentration of the patient to at least one of the patient and the monitor.

15. The system of claim 13, wherein the glucose sensor is positioned to measure a chemical composition of the skin of the patient to determine a glucose concentration.

16. The system of claim 13, wherein the glucose sensor comprises an electrode that is implanted under the skin of the patient to measure the glucose values.

17. The system of claim 13, wherein the at least one medication infusion pump is one of a wearable medication infusion pump or an implantable medication infusion pump.

18. The system of claim 13, wherein the at least one medication infusion pump is integral the peritoneal dialysis system.

19. The system of claim 13, wherein the control system can be programmed to provide automated administration of one of glucose or insulin by the at least one medication infusion pump in response to an incidence of hypoglycemia or an incidence of hyperglycemia.

20. The system of claim 13, the control system programmed to provide an alert if the glucose concentration is outside of a specified range.

21-22. (canceled)
